(19) United States
(12) Patent Application Publication
(10) Pub. No.: US 2023/0201548 A1
(43) Pub. Date: Jun. 29, 2023

(54) CATHETER, BALLOON EXPANSION METHOD, BALLOON COMPRESSION METHOD

(71) Applicant: JAPAN LIFELINE CO., LTD., Tokyo (JP)

(72) Inventors: Ryo SEKI, Tokyo (JP); Daisuke YOSHIKAWA, Tokyo (JP); Yasuhiro OKAWA, Tokyo (JP); Reo TAKEDA, Tokyo (JP); Takuya MASUDA, Tokyo (JP)

(21) Appl. No.: 18/086,142

(22) Filed: Dec. 21, 2022

(30) Foreign Application Priority Data
Dec. 23, 2021 (JP) 2021-209536
Sep. 27, 2022 (JP) 2022-153620

(51) Int. Cl. A61M 29/02

(52) U.S. Cl. CPC A61M 29/02; A61M 2025/1084

(57) ABSTRACT

A balloon catheter includes a shaft inserted into a body; a balloon attached to the shaft on a distal end side and is expandable by an expansion fluid supplied from a side of a proximal end of the shaft; an elastic band wound around an outer periphery of an intermediate portion between a distal end portion and a proximal end portion of the balloon , and is configured to limit expansion of the balloon at the intermediate portion; and a covering member covering an outer periphery of the elastic band. The balloon is expandable to a maximum expansion diameter from a folded state, in accordance with pressure of the expansion fluid, the elastic band is expandable beyond the secured diameter, by being elastically deformed by the balloon expanding, and the covering member is expandable by being elastically deformed by the balloon or the elastic band expanding.

### TECHNICAL FIELD

The present disclosure relates to a catheter, in particular, a balloon catheter including a balloon that can be expanded in a body.

### BACKGROUND ART

A catheter is a medical tube that is inserted into a body for inspection or treatment. In particular, a catheter including a balloon that can be expanded in a body is referred to as a balloon catheter, and is used for dilating a dilatation target part and a constricted part in: a tubular organ in a body such as blood vessels, trachea, gastrointestinal tract, common bile duct, and pancreatic ductus; a connection part (inlet and outlet) between these; a hole formed in the body for inspection or treatment (a hole that is punctured into the common bile duct from the stomach and the duodenal bulb for example); and the like.

In Patent Document 1, an elastic band portion is provided on the outer periphery of an intermediate portion of the balloon in order to reliably dilate the constricted part. Expansion of the balloon starts from shoulder portions on both sides of the band portion. As a result, the band portion between both shoulder portions forms a constricted waist portion. The constricted part that is the dilatation target is supported from both sides by the shoulder portions that have been expanded, and thus can stay at a position facing the waist portion. When the balloon further expands in this state, the constricted part is reliably dilated by the band portion that elastically deforms for the dilatation.

### CITATION LIST

### Patent Document

Patent Document 1: JP 2014-124264 A

### SUMMARY OF INVENTION

### Technical Problem

The balloon that is no longer required once the constricted part has been dilated is compressed to be pulled out from the body together with a shaft. In this process, the band portion that has been elastically deformed and expanded is also compressed. If the band portion has low elasticity, the band portion cannot be sufficiently compressed. This may result in slack produced between the band portion and the balloon that has been fully compressed. In the worst case, the slacked band portion may fall off the balloon and the shaft, to remain in the body. If the elasticity of the band portion is set to be high to reliably prevent the band portion from falling off, the elastic deformability of the band portion is compromised. As a result, the required function of the band portion (and the balloon) to dilate the constricted part is difficult to provide.

The present disclosure has been made in view of such circumstances, and an object thereof is to provide a catheter that can reliably dilate a dilatation target part.

### Solution to Problem

A catheter according to an aspect of the present disclosure for solving the problem described above includes: a shaft to be inserted into a body; a balloon that is attached to the shaft on a distal end side and is expandable by a fluid supplied from a side of a proximal end of the shaft; a securing member that is wound around an outer periphery of an intermediate portion between a distal end portion and a proximal end portion of the balloon, and is configured to limit expansion of the balloon at the intermediate portion; and a covering member that covers an outer periphery of the securing member.

According to this aspect, the balloon expanding first on both sides of the securing member can reliably hold a dilatation target part or a constricted part that is the dilatation target at a position facing the securing member. The covering member covers the securing member so that the balloon can be prevented from falling off the shaft, when the balloon is compressed at the time of taking out the catheter.

Another aspect of the present disclosure relates to a method of expanding a balloon. The method is a method of expanding a balloon in a catheter including a shaft to be inserted into a body, the balloon that is attached to the shaft on a distal end side and is expandable by a fluid supplied from a side of a proximal end of the shaft, a securing member that is wound around an outer periphery of an intermediate portion between a distal end portion and a proximal end portion of the balloon, and is configured to limit expansion of the balloon at the intermediate portion, and a covering member that covers an outer periphery of the securing member, the method including reducing, when the balloon is expanded together with the securing member and the covering member by supplying a fluid into the balloon, a rate of expansion of the intermediate portion around which the securing member is wound.

Yet another aspect of the present disclosure relates to a catheter. The catheter includes: a shaft to be inserted into a body; a balloon that is attached to the shaft on a distal end side and is expandable by a fluid supplied from a side of a proximal end of the shaft; and a covering member that covers an outer periphery of the balloon, and has a large thickness at an intermediate portion between a distal end portion and a proximal end portion of the balloon compared with other portions.

Yet another aspect of the present disclosure relates to a catheter. The catheter includes: a shaft to be inserted into a body; a balloon that is attached to the shaft on a distal end side and is expandable by a fluid supplied from a side of a proximal end of the shaft; and a visually recognized portion that is provided in an intermediate portion between a distal end portion and a proximal end portion of the balloon, and is visually distinguishable from other portions of the balloon.

According to this aspect, an operator of the catheter can reliably bring the intermediate portion of the balloon provided with the visually recognized portion close to the dilatation target part, while watching the visually recognized portion.

Yet another aspect of the present disclosure relates to a catheter. The catheter includes: a shaft to be inserted into a body; and a balloon that is attached to the shaft on a distal end side and includes a first balloon and a second balloon expandable by a fluid supplied from a side of a proximal end of the shaft, the first balloon being provided closer to an outer side in a direction of the expansion than the second balloon, and being softer than the second balloon.

According to this aspect, the balloon has at least dual structure in which the soft first balloon on the outer side expands while conforming to the shape of the dilatation target part such as the constricted part, whereby the balloon can be positioned with respect to the dilatation target part, and the hard second balloon on the inner side can reliably dilate the dilatation target part.

Yet another aspect of the present disclosure relates to a catheter. The catheter includes: a shaft to be inserted into a body; and a balloon that is attached to the shaft on a distal end side, is expandable by a fluid supplied from a side of a proximal end of the shaft, and includes an end portion securing structure that limits, after expansion into a constricted shape having a constricted portion in an intermediate portion between a distal end portion and a proximal end portion, the expansion in the distal end portion and the proximal end portion.

According to this aspect, the balloon can be reliably positioned with respect to the dilatation target part, with the constricted portion formed in the intermediate portion at the time when the balloon expands. When the dilatation target part is dilated with the constricted portion further expanded by the fluid, the end portion securing structure can effectively prevent the distal end portion and the proximal end portion on the distal and proximal end sides of the constricted portion from excessively expanding.

Yet another aspect of the present disclosure relates to a catheter. The catheter includes: a shaft to be inserted into a body; and a balloon that is attached to the shaft on a distal end side, is expandable by a fluid supplied from a side of a proximal end of the shaft, and includes a compression structure configured to cause an intermediate portion between a distal end portion and a proximal end portion to compress before the distal end portion and the proximal end portion compress, at a time of compression in which the fluid is discharged.

According to this aspect, the balloon can be reliably compressed by the compression structure provided to the intermediate portion of the balloon.

Yet another aspect of the present disclosure relates to a method of compressing a balloon of a catheter. The method is a method of compressing a balloon of a catheter including a shaft to be inserted into a body, and the balloon that is attached to the shaft on a distal end side, is expandable by a fluid supplied from a side of a proximal end of the shaft, and includes a compression structure configured to cause an intermediate portion between a distal end portion and a proximal end portion to compress before the distal end portion and the proximal end portion compress, at a time of compression in which the fluid is discharged. The method includes increasing a rate of compression of the intermediate portion provided with the compression structure, when compressing the balloon by discharging the fluid in the balloon.

Yet another aspect of the present disclosure relates to a catheter. The catheter includes: a shaft to be inserted into a body; a balloon that is attached to the shaft on a distal end side and is expandable by a fluid supplied from a side of a proximal end of the shaft; and a securing member that is wound around an outer periphery of an intermediate portion between a distal end portion and a proximal end portion of the balloon, is configured to limit expansion of the balloon at the intermediate portion, and includes a securing relaxation structure that relaxes securing in at least one of a distal end portion and a proximal end portion of the securing member.

According to this aspect, the securing member (and the balloon) can be reliably positioned with respect to the dilatation target part, because the constricted portion is not only formed in the balloon but is also formed in the securing member due to the securing being relaxed in the end portions of the securing member.

### Advantageous Effects of Invention

With a catheter and the like of the present disclosure, a dilatation target part can be reliably dilated.

### BRIEF DESCRIPTION OF DRAWINGS

FIG. 1 schematically illustrates an overview of EPBD with a papilla being the dilatation target.

FIG. 2 is a side view illustrating the outer appearance of a balloon catheter according to the present embodiment.

FIG. 3 is a side view illustrating the outer appearance of the balloon catheter according to the present embodiment.

FIGS. 4A to 4D are cross-sectional views of the balloon catheter according to the present embodiment.

FIGS. 5A to 5C schematically illustrate an expanded form of a balloon and an elastic band.

FIGS. 6A to 6C schematically illustrate a method of manufacturing the balloon catheter.

FIGS. 7a to 7C cross-sectional views schematically illustrating a plurality of modifications of a covering member.

FIGS. 8A to 8C schematically illustrate a plurality of examples of a cross section taken along line E-E in FIG. 7.

FIGS. 9A to 9C schematically illustrate a modification of the balloon.

FIG. 10 is a perspective view schematically illustrating a modification of a shaft with which expansion fluid can be supplied to a first balloon and a second balloon at the same timing or different timings.

FIGS. 11A and 11B are cross-sectional views schematically illustrating a modification of the balloon catheter.

FIG. 12 schematically illustrates a modification of an elastic band.

### DESCRIPTION OF EMBODIMENTS

Hereinafter, embodiments for carrying out the present disclosure will be described in detail with reference to the drawings. In the description or drawings, the same or equivalent constituent elements, members, and processing operations are denoted by the same reference numerals, and overlapping descriptions are omitted. The scales and shapes of the illustrated parts are set for convenience to facilitate the explanation and should not be construed as limiting unless otherwise specified. The embodiments are illustrative and do not limit the scope of the present disclosure in any way. Not all features or combinations of features described in the embodiments are essential to the present invention.

A catheter of the present disclosure can be used for dilatation of a dilatation target part and a constricted part of any part in a body (for example, tubular organ in a body such as blood vessels, trachea, gastrointestinal tract, common bile duct, and pancreatic ductus; a connection part between these; a hole formed in the body for inspection or treatment), but in the present embodiment, a description will be given using an endoscopic papillary balloon dilatation (EPBD) for dilating a papilla (major duodenal papilla or duodenal papilla) that is a dilatation target part.

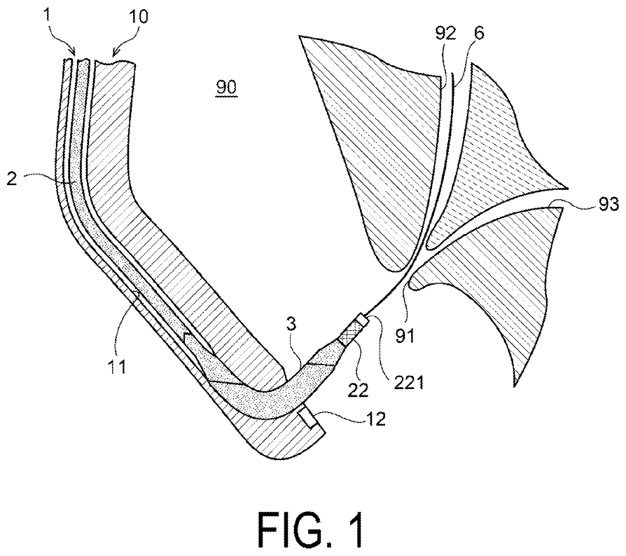

FIG. 1 schematically illustrates an overview of EPBD with a papilla 91 being the dilatation target. An endoscope 10 including a forceps channel 11 and a camera 12 is inserted in a duodenum 90 via the mouth. A balloon 3 is attached to a distal end side (the duodenum 90 side or the in body side) of a tubular shaft 2 of a balloon catheter 1 inserted into the body through the forceps channel 11. The balloon 3 can be expanded using an expansion fluid obtained by mixing contrast agent as appropriate to sterile distilled water or saline, and supplied from the proximal end side (the mouth side or the out of body side) of the shaft 2. Other types of liquid or gas such as air may be used as the expansion fluid, if necessary for the purpose of the inspection or the treatment.

A guidewire 6 with a small diameter is inserted in advance, through the forceps channel 11, in a common bile duct 92 and/or a pancreatic duct 93 that is the target site of the inspection or the treatment or a route leading to the target site. While the papilla 91 serving as the dilatation target part or an opening part is provided between the duodenum 90 and the common bile duct 92 and the pancreatic duct 93, the guidewire 6 has a diameter sufficiently smaller than the opening diameter of the papilla 91, and thus can enter into the common bile duct 92 or the pancreatic duct 93 through the papilla 91. In this process, an operator of the endoscope 10 and the balloon catheter 1 can safely insert the guidewire 6 into the papilla 91, while checking an image obtained from the camera 12 disposed on a side surface of the endoscope 10 to face the papilla 91.

A long wire lumen (hole), in which the guidewire 6 can be inserted, is formed through the internal of the tubular shaft 2 of the balloon catheter 1, that is, from a proximal end portion to a distal end portion. In a state where the guidewire 6 is inserted into the common bile duct 92 or the pancreatic duct 93 through the papilla 91, the distal end portion of the wire lumen in the shaft 2 is inserted from the proximal end portion that is on the out of body side of the guidewire 6. Thus, the balloon 3 at the distal end portion of the shaft 2 guided by the guidewire 6 moves to the papilla 91. In the state illustrated, the shaft 2 is further moved forward along the guidewire 6, and the balloon 3 that has reached the position of the papilla 91 is expanded by the expansion fluid. Thus, the papilla 91 is dilated from the inner side. In this way, the papilla 91, which is usually constricted by the sphincter Oddi (or biliary pancreatic ampulla sphincter) is dilated. Thus, a common bile duct stone formed in the common bile duct 92 can be effectively taken out through the papilla 91 for example.

The balloon 3 that is no longer require after the papilla 91 has been dilated is compressed by discharging the expansion fluid to the out of body side, and then is taken out from the body together with the shaft 2 through the forceps channel 11. After the balloon catheter 1 has been thus taken out from the body, a medical instrument such as another forceps or cholangioscope for another medical procedure, such as moving the common bile duct stone from the papillary 91 into the duodenum 90 or to the outside of the body, is inserted into the dilated papilla 91 through the guidewire 6 if necessary.

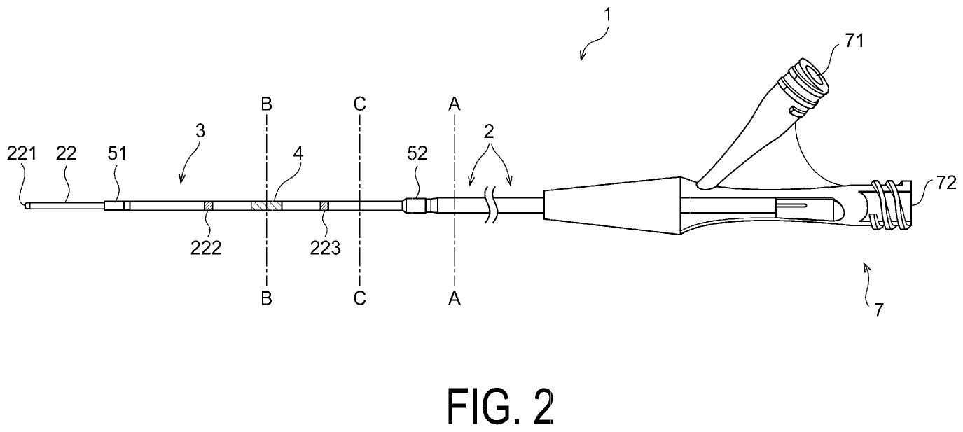

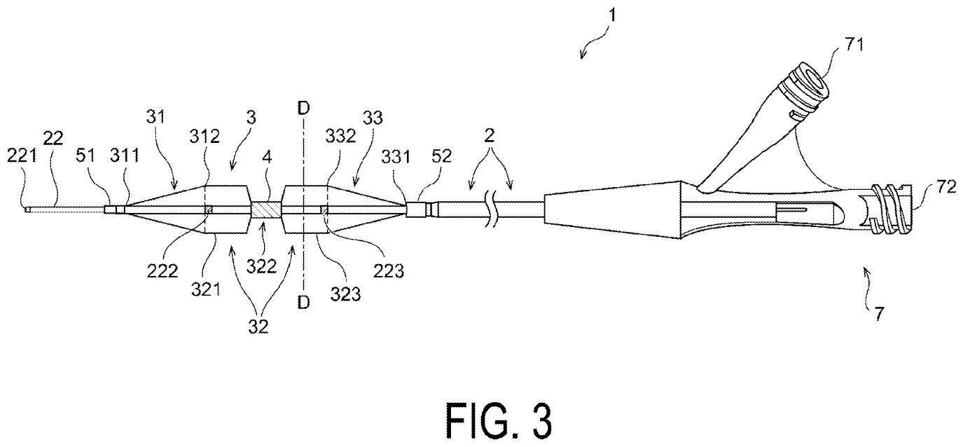

FIGS. 2 and 3 are side views illustrating the outer appearance of the balloon catheter 1 according to the present embodiment. In these drawings, the balloon 3 is attached to a distal end portion (left end portion) of the shaft 2 of an elongated tube shape inserted into the body from the right side (out of body side) toward the left side (in body side). FIG. 2 illustrates the balloon 3 in a completely compressed state. FIG. 3 illustrates the balloon 3 in a partially expanded state which is a state between the completely compressed state and a completely expanded state.

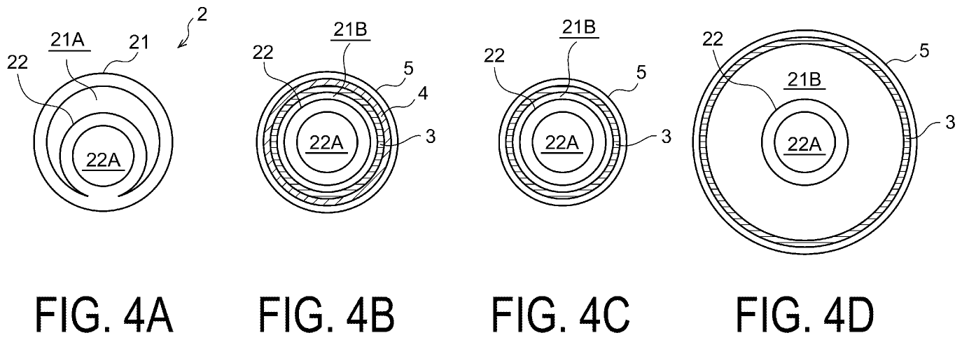

A cross section of the shaft 2 on the proximal end side, specifically, a portion of the shaft 2 where the balloon 3 is not attached is partitioned into two lumens, that is, a balloon expansion lumen 21A and a wire lumen 22A as can be seen in FIG. 4A illustrating a cross section taken along line A-A in FIG. 2. In the illustrated example, an expansion fluid tube 21 having a large diameter and a guidewire tube 22 that has a small diameter and is contained on the inner side of the expansion fluid tube 21 are integrally formed. The balloon expansion lumen 21A is a space defined by the inner periphery of the expansion fluid tube 21 and the outer periphery of the guidewire tube 22. The wire lumen 22A is a space defined by the inner periphery of the guidewire tube 22.

The balloon expansion lumen 21A communicates with a balloon expansion port 71 of a manifold 7 provided to the proximal end portion (the right end portion in FIG. 2 and FIG. 3) on the out of body side of the shaft 2. The expansion fluid supplied and discharged through the balloon expansion port 71 flows into and out from the balloon 3 through the balloon expansion lumen 21A. Specifically, when the expansion fluid is supplied from the balloon expansion port 71, the expansion fluid flows into the balloon 3 through the balloon expansion lumen 21A. Thus, the balloon 3 expands. On the other hand, when the expansion fluid is discharged from the balloon expansion port 71, the expansion fluid flows out from the balloon 3 through the balloon expansion lumen 21A. Thus, the balloon 3 is compressed.

Although not elaborated in the figure, the expansion fluid tube 21 forming the balloon expansion lumen 21A has a tapered shape, and has an opening end with a small diameter inserted in an internal space 21B of the balloon 3. Through this opening end, the expansion fluid flows between the expansion fluid tube 21 (balloon expansion lumen 21A) and the internal space 21B of the balloon 3. The internal space 21B of the balloon 3 is illustrated as a space defined by the inner periphery of the balloon 3 and the outer periphery of the guidewire tube 22, in FIG. 4B, FIG. 4C, and FIG. 4D illustrating cross sections taken along lines B-B, C-C, and D-D in FIG. 2 or FIG. 3 respectively.

The wire lumen 22A communicates with a guidewire port 72 of the manifold 7 provided to the proximal end portion of the shaft 2 (the right end portion in FIG. 2 and FIG. 3) on the out of body side. The guidewire tube 22 forming the wire lumen 22A is formed through the internal space 21B of the balloon 3 toward the distal end side (left end side in FIG. 2 and FIG. 3), unlike the expansion fluid tube 21 terminating within the internal space 21B of the balloon 3. As described above, the long wire lumen 22A, in which the guidewire 6 can be inserted, is formed through the internal of the tubular shaft 2 of the balloon catheter 1, that is, from the guidewire port 72 in the proximal end portion to an opening end 221 in the distal end portion. As described above with reference to FIG. 1, in a state where the guidewire 6 is inserted into the common bile duct 92 or the pancreatic duct 93 through the papilla 91, the opening end 221 of the guidewire tube 22 that is the distal end portion of the wire lumen in the wire lumen 22A is inserted from the proximal end portion that is on the out of body side of the guidewire 6. Thus, the balloon 3 at the distal end portion of the shaft 2 guided by the guidewire 6 moves to the papilla 91.

A configuration of the balloon catheter 1 on the distal end side where the balloon 3 is provided will be described more in detail. As illustrated in FIG. 4B and FIG. 4C, the balloon 3 folded in the completely compressed state is attached to the outer periphery of the guidewire tube 22 forming the distal end portion of the shaft 2. The internal space 21B between the inner periphery of the balloon 3 in the completely compressed state and the outer periphery of the guidewire tube 22 is illustrated in an exaggerated manner in FIG. 4B and FIG. 4C. The space is actually small enough to be ignorable. Thus, the inner periphery of the balloon 3 in the completely compressed state and the outer periphery of the guidewire tube 22 are in contact with each other almost with no gap in between.

As illustrated in FIG. 3, the balloon 3 includes three portions different from each other in the expanded form or expansion mode that are a distal end side tapered portion 31, an intermediate portion 32, and a proximal end side tapered portion 33 in this order from the distal end toward the proximal end. The distal end side tapered portion 31 is formed to have a tapered shape with the maximum expansion diameter increasing toward the intermediate portion 32 from a distal end portion 311 of the balloon 3, the diameter of which is substantially the same as that of the outer circumference of the guidewire tube 22. The proximal end side tapered portion 33 is formed to have a tapered shape with the maximum expansion diameter increasing toward the intermediate portion 32 from a proximal end portion 331 of the balloon 3, the diameter of which is substantially the same as that of the outer circumference of the guidewire tube 22 (the shaft 2 further including the expansion fluid tube 21 having a small diameter).

In the illustrated example, the length of the distal end side tapered portion 31 and the length of the proximal end side tapered portion 33 are substantially the same but may be significantly different from each other, in a direction connecting the distal end (opening end 221) and the proximal end (guidewire port 72) of the shaft 2 (hereinafter, referred to as an axial direction, a longitudinal direction, or a left and right direction, and a dimension in such a direction is referred to as the length). An expansion diameter of the distal end side tapered portion 31 and an expansion diameter of the proximal end side tapered portion 33 at points equidistant respectively from the distal end portion 311 and the proximal end portion 331 are substantially the same in the illustrated example, but may be significantly different from each other in any direction orthogonal to the axial direction (hereinafter, referred to as a radial direction, an expansion direction, or an orthogonal direction, and a dimension in such a direction is referred to as the expansion diameter). In the illustrated example where the maximum expansion diameter of a proximal end portion 312 of the distal end side tapered portion 31 and the maximum expansion diameter of a distal end portion 332 of the proximal end side tapered portion 33 are substantially the same, the intermediate portion 32 coupling these portions in the axial direction serves as a straight tube portion having a substantially uniform maximum expansion diameter.

As illustrated in FIGS. 3 and 4B, an annular elastic band 4 serving as a securing member is wound around at least part of the outer periphery of the intermediate portion 32, to limit the expansion of the balloon 3 in the intermediate portion 32. In the illustrated example, a band shaped elastic band 4 having a uniform width or length in the axial direction is wound around the outer periphery of a center portion 322 of the intermediate portion 32. The elastic band 4 does not limit the expansion of a distal end side straight tube portion 321 closer to the distal end side than the center portion 322 in the intermediate portion 32. Thus, the distal end side straight tube portion 321 can easily expand to have a substantially uniform maximum expansion diameter. Similarly, the elastic band 4 does not limit the expansion of a proximal end side straight tube portion 323 closer to the proximal end side than the center portion 322 in the intermediate portion 32. Thus, the proximal end side straight tube portion 323 can easily expand to have a substantially uniform maximum expansion diameter. On the other hand, the elastic band 4 limits the expansion of the center portion 322 of the intermediate portion 32. Thus, the expansion diameter of the center portion 322 in a case where the pressure of the expansion fluid in the balloon 3 (internal space 21B) is less than a predetermined value (less than 2 atm for example) is substantially (at least 20% for example) smaller than the expansion diameter of the distal end side straight tube portion 321 and the proximal end side straight tube portion 323 that can be easily expanded.

In the description below, the initial diameter of the inner periphery of the elastic band 4 before the introduction of the expansion fluid is also referred to as a secured diameter. Before the expansion fluid is introduced, the outer periphery of the center portion 322 of the balloon 3 in the completely compressed state is in contact with the inner periphery of the elastic band 4, and thus the initial diameter of the balloon 3 (center portion 322) is also referred to as the secured diameter below. When the expansion fluid is introduced into the balloon 3 (internal space 21B), the elastic band 4 starts to elastically deform to be expanded from the secured diameter or the initial diameter. Still, as long as the pressure of the expansion fluid is at least in a range smaller than the predetermined value, the distal end side straight tube portion 321 and the proximal end side straight tube portion 323 on both sides expand at a higher rate. Thus, the balloon 3 expands to be in a dumbbell shape with the center portion 322 where the elastic band 4 is provided being constricted relative to the distal end side straight tube portion 321 and the proximal end side straight tube portion 323. The expansion diameter varies among positions of the elastic band 4 in the longitudinal direction. Specifically, the expansion diameter in both end portions expanding together with the distal end side straight tube portion 321 and the proximal end side straight tube portion 323 that easily expand is larger than that in the center portion.

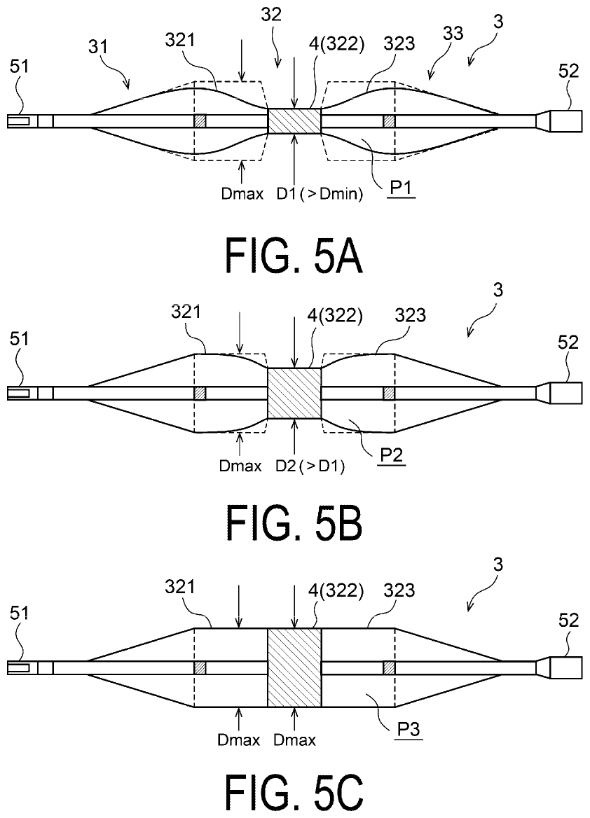

The elastic band 4 formed by any elastomeric material, such as rubber, is elastically deformed in response to the expansion of the balloon 3 when the expansion fluid is introduced into the balloon 3 (internal space 21B), to be expanded to have a diameter larger than the secured diameter. FIGS. 3 and 5A illustrate a state immediately after the elastic band 4 has started to elastically deform, with the expansion fluid introduced into the balloon 3. In this state, the distal end side straight tube portion 321 and the proximal end side straight tube portion 323 the expansion of which is not limited by the elastic band 4 largely expand beyond the elastic band 4. On the other hand, the diameter of the center portion 322 the expansion of which is limited by the elastic band 4 is limited to an expanded diameter D1 slightly larger than a secured diameter Dmin of the elastic band 4 (the inner diameter of the elastic band 4 in FIG. 2). In this state, pressure P1 of the expansion fluid in the balloon 3 (internal space 21B) is lower than the predetermined value described above. Thus, the expanded diameter D1 is smaller than a maximum expansion diameter Dmax of the distal end side straight tube portion 321 and the proximal end side straight tube portion 323 by at least 20% (D1<0.8×Dmax).

As an example of dimensions, the maximum expansion diameter Dmax is from 7 mm to 9 mm (preferably about 8 mm), and the secured diameter Dmin is from 1.5 mm to 2.5 mm (preferably about 2 mm). The diameter of the folded balloon 3 in the completely compressed state in FIG. 2 is from 1.5 mm to 2.5 mm (preferably about 1.8 mm). The diameter of the shaft 2 closer to the proximal end side than the balloon 3 is from 1.5 mm to 3 mm (preferably from 2 mm to 2.5 mm).

As illustrated in FIG. 3 and FIG. 5A, the expansion of the balloon 3 results in formation of a constricted portion having a small diameter (D1) in the center portion 322 between the distal end side straight tube portion 321 and the proximal end side straight tube portion 323 expanded beyond the elastic band 4. When the balloon 3 is expanded as illustrated in FIG. 3 and FIG. 5A in a state where the center portion 322 is in the vicinity of the papilla 91 (FIG. 1) that is the dilatation target, the papilla 91 supported from both sides by the distal end side straight tube portion 321 and the proximal end side straight tube portion 323 expanded beyond the elastic band 4 can stay at a position facing the constricted portion (center portion 322). With the constricted portion, the papilla 91 can be effectively supported from both sides when the pressure of the expansion fluid is from 0.5 atm to 1.5 atm (preferably about 1.0 atm).

When the pressure of the expansion fluid in the balloon 3 (internal space 21B) is increased to a predetermined value P2, the elastic band 4 expands to an expansion diameter D2 as in FIG. 5B. Furthermore, the distal end side of the distal end side straight tube portion 321 and the proximal end side of the proximal end side straight tube portion 323 expand to the maximum expansion diameter Dmax. This expansion diameter D2 of the center portion 322 is smaller than the maximum expansion diameter Dmax of the distal end side straight tube portion 321 and the proximal end side straight tube portion 323 by 20% (D2=0.8×Dmax). The maximum expansion diameter Dmax slightly increases in accordance with the pressure of the expansion fluid due to the elasticity of the balloon 3 itself, but such an increase is practically ignorable. When the pressure of the expansion fluid is further increased from that in the state illustrated in FIG. 5B (to be from 4 atm to 5 atm for example), as illustrated in FIG. 5C, the center portion 322 (elastic band 4) may be expanded to the maximum expansion diameter Dmax of the balloon 3 (intermediate portion 32) which is the same as those of the distal end side straight tube portion 321 and the proximal end side straight tube portion 323. At least a difference between the expansion diameter of the elastic band 4 or the center portion 322 and the expansion diameter (maximum expansion diameter) Dmax of the distal end side straight tube portion 321 and the proximal end side straight tube portion 323 becomes smaller than 20%.

In this state, the intermediate portion 32 including the center portion 322 around which the elastic band 4 is wound is in a straight tube shape expanded to the maximum expansion diameter Dmax that is substantially uniform. With the elastic band 4 (center portion 322) thus elastically deforming to expand beyond the secured diameter Dmin in response to the expansion of the balloon 3, the papilla 91 is reliably dilated. When the papilla 91 can be sufficiently dilated with the elastic band 4 expanding to any diameter D2 larger than the secured diameter Dmin and smaller than the maximum expansion diameter Dmax as illustrated 5B, the state illustrated in FIG. 5B may be the final expanded state of the balloon 3, and thus the elastic band 4 may not be expanded to the maximum expansion diameter Dmax as illustrated in FIG. 5C. The pressure of the expansion fluid in the balloon 3 (internal space 21B) achieving the final expanded state is defined as a recommended expansion pressure, and is, for example, from 3 atm to 5 atm (preferably about 4 atm).

To reliably position the balloon 3 to the papilla 91 as described above, a contrast agent mixed in the expansion fluid supplied into the balloon 3 and two contrast markers 222 and 223 provided at positions corresponding to both end portions (312 and 332) of the intermediate portion 32 of the balloon 3 on the outer periphery of the guidewire tube 22 are used. Using a contrast image captured during the EPBD procedure using X ray or the like, the position of the balloon 3 relative to the papilla 91 and the state of expansion of the balloon 3 can be checked in real time.

To effectively hold the papilla 91 of a normal size at the position facing the constricted portion during such EPBD, the width or the length in the axial direction of the elastic band 4 forming the constricted portion is preferably set to be from 1 mm to 10 mm. To reduce the risk of balloon 3, inserted into the common bile duct 92 through the papilla 91, pressing the pancreatic duct 93 to cause inflammation, the length of the balloon 3 is preferably set to be short as possible. In has been known that the balloon 3 needs to have a long length to prevent the expanded balloon 3 from slipping out from the papilla 91. Still, with the balloon catheter 1 of the present embodiment, the constricted portion formed by the elastic band 4 can reliably hold the papilla 91, and thus the balloon 3 can have a short length. Specifically, the length of the intermediate portion 32 (straight tube portion) that is the effective length of the balloon 3 may be set to be from 10 mm to 40 mm. The length of the intermediate portion 32 of the balloon 3 is preferably two to eight times longer than the length of the elastic band 4. For example, when the length of the elastic band 4 is 5 mm, the length of the intermediate portion 32 of the balloon 3 is preferably from 10 mm(×2) to 40 mm(×8).

In the above description, the elastic band 4 which is a single elastic member having an annular and band shape surround at least part (center portion 322) of the outer periphery of the intermediate portion 32 is described as an example of the securing member. However, the securing member is not limited to this. For example, the securing member may be a plurality of elastic rings wound around at least part of the outer periphery of the intermediate portion 32, or may be a coil spring made of a nickel titanium alloy (NiTi) or the like spirally wound around at least part of the outer periphery of the intermediate portion 32. These elastic members may be any member applying significant elastic force in a compression direction when the expansion occurs due to the pressure of the expansion fluid, and does not necessarily need to completely return to the original state (secured diameter or initial diameter) after the expansion fluid has been discharged. Thus, the elastic member may remain somewhat deformed (thus, plastic deformation) after the expansion fluid has been discharged. The securing member may be a member having no or limited elasticity. For example, a member with plasticity made of metal or the like that can be expanded beyond the secured diameter by being plastically deformed by the balloon 3 expanding, but cannot be compressed after the expansion may be used as the securing member. Furthermore, a rigid member that is practically not deformable, maintains the secured diameter as long as the pressure of the expansion fluid in the balloon 3 is lower than a predetermined value, and breaks once the pressure of the expansion fluid in the balloon 3 reaches or exceeds the predetermined value may be used as the securing member.

The securing member that is the elastic member or the member with plasticity may break when the pressure of the expansion fluid in the balloon 3 increases after the elastic deformation or plastic deformation of the securing member to a predetermined diameter (the diameter D2 in FIG. 5B for example). When the securing member is thus removed, the center portion 322 automatically expands to the maximum expansion diameter Dmax as illustrated in FIG. 5C (with the elastic band 4 broken). As a result, the intermediate portion 32 as a whole expands to the substantially uniform maximum expansion diameter Dmax to be a straight tube shape. A covering member 5 described below covers the securing member, including the member with plasticity after the plastic deformation or the rigid member that has been broken, from the outer periphery, whereby the securing member can be prevented from falling off the balloon 3 and the shaft 2. Because the securing member is thus prevented from falling off in the body, the securing member can also be formed using a material that is not generally used for medical purposes. The securing member (the elastic band 4 or the like) may be formed using a material that does not transmit X-rays, to have a contrast function similar to that of the contrast markers 222 and 223 provided on the outer periphery of the guidewire tube 22.

The securing member that remains in the covering member 5 may also function as an alignment marker (visually recognized portion) visible via the camera 12 of the endoscope 10. Such a visually recognized portion is provided in the intermediate portion 32 (the center portion 322 in particular) of the balloon 3, and has a feature that is visually distinguishable from other portions of the balloon 3 (the distal end side straight tube portion 321, the proximal end side straight tube portion 323, the distal end side tapered portion 31, and the proximal end side tapered portion 33 for example). For example, the elastic band 4 (securing member) that is the visually recognized portion has a color and/or a pattern different from those of the other portions of the balloon 3. For example, the balloon 3 is typically colorless or white and has no pattern. Thus, the elastic band 4 is provided with an achromatic color other than white (grey or black) or any chromatic color and/or is provided with any pattern to function as the visually recognized portion visually distinguishable from the balloon 3. The operator of the balloon catheter 1 can safely and reliably operate the balloon catheter 1, while visually checking the elastic band 4 serving as the visually recognized portion distinguishable from the balloon 3 and the target site such as the papilla 91.

The visually recognized portion is not limited to the elastic band 4 that is the securing member, and may be formed in the balloon 3 and/or the covering member 5. When the visually recognized portion is formed as part of the balloon 3, the intermediate portion 32 (the center portion 322 in particular) where the visually recognized portion is to be formed may be provided with a color and/or a pattern different from those in the other portions. Specifically, a portion of the balloon 3 to be the visually recognized portion may have a material or property different from those in other portions, to a have difference in color, pattern, or the like to be visually distinguishable from the other portions. When the visually recognized portion is formed in the covering member 5, a position corresponding to the intermediate portion 32 (the center portion 322 in particular) of the balloon 3 where the visually recognized portion is to be formed may be provided with a color and/or a pattern different from those in the other portions. The visually recognized portion may be applied to the balloon catheter 1 without the elastic band 4 and/or the covering member 5. In this case, the visually recognized portion is preferably formed in the balloon 3 as described above, but an annular guide band (which does not function as the securing member) may be wound around the outermost periphery of the intermediate portion 32 (the center portion 322 in particular) of the balloon 3 in addition to or instead of the elastic band 4.

The covering member 5 covers the outer periphery of the elastic band 4 as illustrated in FIG. 4B and covers the outer periphery of the balloon 3 as illustrated in FIG. 4C and FIG. 4D. The thickness of the covering member 5 (not expanded) in the radial direction is, for example, approximately 50 μm, and the thickness of the elastic band 4 (not expanded) in the radial direction is, for example, approximately 100 μm. The covering member 5 is a tube having an elongated tubular shape formed by any elastic material such as polyurethane. In FIG. 3, the distal end portion of the covering member 5 (not illustrated) is fixed by a short fixing tube 51 made of thermoplastic elastomer or the like, in a state of covering the outer periphery of the guidewire tube 22 extending toward the distal end side from the distal end portion 311 of the balloon 3. The proximal end portion of the covering member 5 is fixed by a short fixing tube 52 made of thermoplastic elastomer or the like, in a state of covering the outer periphery of the shaft 2 extending toward the proximal end side from the proximal end portion 331 of the balloon 3. The fixing tube 51 has a smaller diameter than the fixing tube 52, due to a difference in diameter in the guidewire tube 22 having a small diameter on the distal end side and the shaft 2 having a large diameter on the proximal end side.

As described above, both end portions of the covering member 5 longer than the elastic band 4 and the balloon 3 are fixed to the outer periphery of the shaft 2 (including the guidewire tube 22) at portions closer to the distal end side and the proximal end side than the elastic band 4. The covering member 5 may be shorter than the balloon 3, and have both end portions fixed to the outer periphery of the balloon 3 at portions closer to the distal end side and the proximal end side than the elastic band 4. For example, both end portions of the covering member 5 may be fixed to the outer periphery of the distal end side tapered portion 31 and the proximal end side tapered portion 33, or fixed to the outer periphery of the distal end side straight tube portion 321 and the proximal end side straight tube portion 323. In some cases, an electrode for medical procedure, measurement, or the like or a distal end chip (a member where the opening end 221 is formed) forming the distal end portion of the balloon catheter 1 or the shaft 2 may be provided closer to the distal end side than the balloon 3 in the balloon catheter 1. The covering member 5 may be fixed to the outer periphery of (part of) such electrode or distal end chip.

With these configurations, the covering member 5 covers the outer periphery of the balloon 3 not wound by the elastic band 4 as well as the outer periphery of the elastic band 4, so that the elastic band 4 and other securing members can be prevented from falling from the balloon 3. Additionally, in the illustrated example, the covering member 5 covers the outer periphery of the shaft 2 (including the guidewire tube 22) where the cover 3 is not attached as well as the outer periphery of the elastic band 4 and the balloon 3. Thus, not only the elastic band 4 and other securing members can be prevented from falling off the balloon 3 and the shaft 2, but also the balloon 3 can be effectively compressed by the elasticity of the covering member 5 when the balloon catheter 1 is taken out.

As described above, the covering member 5 has both end portions fixed to the outer periphery of the shaft 2 and/or the balloon 3 at both ends, but does not have portions other than both end portions fixed to the outer periphery of the balloon 3 and/or the elastic band 4. Thus, as illustrated in FIG. 4B, the elastic band 4 covered by the covering member 5 from the outer side is not bonded to the inner periphery of the covering member 5. Similarly, the elastic band 4 is not bonded to the outer periphery of the balloon 3 (center portion 322) on the inner side. Still, the elastic band 4 is pressed against the balloon 3 from the outer side due to the elasticity of the covering member 5 to be able to stay at a predetermined position (center portion 322), and thus can be also prevented from falling off the balloon 3. In Patent Document 1, a band portion is welded to a balloon, but in the present embodiment, the elastic band 4 does not need to be bonded to the balloon 3 (and the covering member 5). Thus, the balloon catheter 1 can be manufactured economically. In addition, when the band portion is welded to the balloon as in Patent Document 1, a desired expansion of the balloon and the band portion might be inhibited at the welded portion. In the present embodiment, the elastic band 4 and the balloon 3 are not welded or bonded, so that their desired expansion and can be achieved without inhibiting each other's expansion.

The covering member 5 with elasticity, in the state in FIG. 2 where the balloon 3 is not expanded, presses the balloon 3 and/or the elastic band 4 from the outer side to maintain the completely compressed state of the balloon 3. When the expansion fluid is supplied to the balloon 3 (internal space 21B), the balloon 3 and/or the elastic band 4 expands, and thus the covering member 5 elastically deforms to expand. The initial diameter or the covering diameter (the inner diameter of the covering member 5 at a positioned where the elastic band 4 is not provided as illustrated in FIG. 4C) when the elastic deformation of the covering member 5 starts is smaller than the secured diameter Dmin of the elastic band 4 illustrated in FIG. 4B.

To achieve the expanded form of the balloon 3 and the elastic band 4 as illustrated in FIG. 5, the elastic modulus of the balloon 3 is set to be higher than that of the elastic band 4, and the elastic modulus of the elastic band 4 is set to be higher than that of the covering member 5. Since the elastic modulus (also known as Young's modulus) indicates how low the deformability is, the balloon 3 is less likely to deform than the elastic band 4, and the elastic band 4 less likely to deform than the covering member 5. In other words, the covering member 5, the elastic band 4, and the balloon 3 are in this order in deformability. When the balloon 3 and/or the elastic band 4 on the inner side deforms, the covering member 5 with the highest deformability expands to substantially completely follow its or their expansion.

When the expansion fluid is supplied into the balloon 3 (internal space 21B) in the completely compressed state in FIG. 2, the balloon 3 starts to expand from the folded state, and the elastic band 4 wound around the center portion 322 of the balloon 3 and the covering member 5 covering these also expand. FIG. 5A illustrates a state where the pressure of the expansion fluid in the balloon 3 (internal space 21B) has reached P1. As described above, the balloon 3 is expanded into a dumbbell shape with the distal end side straight tube portion 321 and the proximal end side straight tube portion 323 expanding beyond the center portion 322, and with the center portion 322 limited to the expanded diameter D1 smaller than the maximum expansion diameter Dmax by at least 20%.

When the pressure of the expansion fluid in the balloon 3 (internal space 21B) is further increased to P2(>P1) from the state in FIG. 5A, the elastic band 4 further elastically deforms as illustrated in FIG. 5B, to expand beyond the expanded diameter D1 in FIG. 5A, and the covering member 5 covering the elastic band 4 also expands to follow this expansion. The distal end side tapered portion 31 and the proximal end side tapered portion 33 of the balloon 3 have been expanded to the maximum expansion diameter at the point of FIG. 5A, and thus does not substantially further expand even when the pressure of the expansion fluid rises in FIG. 5B due to high elasticity of the balloon 3. Furthermore, the distal end side of the distal end side straight tube portion 321 and the proximal end side of the proximal end side straight tube portion 323 expand to the maximum expansion diameter Dmax. When the pressure of the expansion fluid in the balloon 3 (internal space 21B) is further increased to P3(>P2) from the state in FIG. 5B, the elastic band 4 expands to the maximum expansion diameter Dmax as illustrated in FIG. 5C, and the elastic band 4 covering the covering member 5 also expands. Even when the pressure of the expansion fluid in the balloon 3 (internal space 21B) further rises from the state illustrated in FIG. 5C, the balloon 3 does not substantially further expand due to its high elasticity.

When the pressure of the expansion fluid in the balloon 3 (internal space 21B) is reduced and/or the expansion fluid is discharged from the balloon expansion port 71 to take out the balloon catheter 1, the balloon 3 is effectively compressed due to the elasticity of the elastic band 4 and/or the covering member 5 and thus can return to a state close to the completely compressed state in FIG. 2 before the expansion. In this case, the elastic band 4 functions as a compression structure that causes the intermediate portion 32 (center portion 322 in particular) of the balloon 3 to compress before the distal end portion (including the distal end side straight tube portion 321) and the proximal end portion (including the proximal end side straight tube portion 323) compress, at the time of compression with the expansion fluid discharged from the balloon 3. In other words, the elastic band 4 serving as the compression structure causes the intermediate portion 32 of the balloon 3 provided with the compression structure to compress at a higher rate than the distal end portion and the proximal end portion. The elasticity of the elastic band 4 expanded to the maximum expansion diameter Dmax as illustrated in FIG. 5C may be reduced from the original elasticity (at the point of FIG. 5A for example). Still, with the remaining elasticity at the point of FIG. 5C, the elastic band 4 causes the intermediate portion 32 to compress first at the time of compression of the balloon 3, so that the balloon 3 as a whole can be effectively compressed while sequentially transitioning to the states in FIG. 5B and FIG. 5A in this order.

As described above, because the balloon 3 is effectively compressed due to elasticity of the elastic band 4 and/or the covering member 5, it is possible to prevent the balloon 3 from becoming stuck in the forceps channel 11 (FIG. 1) of the endoscope 10, and thus the balloon catheter 1 can be easily taken out. Regardless of the state of expansion or compression of the balloon 3, the elastic band 4 is held while being pressed against the balloon 3 from the outer side due to the elasticity of the covering member 5, whereby the elastic band 4 or the other securing members can be effectively prevented from falling off the balloon 3.

In Patent Document 1 involving a risk that the band portion may fall off, the elasticity of the band portion needs to be higher than necessary to prevent the falling. Meanwhile, in the present embodiment, the covering member 5 provides the falling prevention function, and thus the risk of falling off needs not to be taken into consideration when designing the elasticity, dimension, the shape, and the like of the elastic band 4. Thus, the design can be exclusively optimized for achieving the desired expanded form as illustrated in FIG. 5.

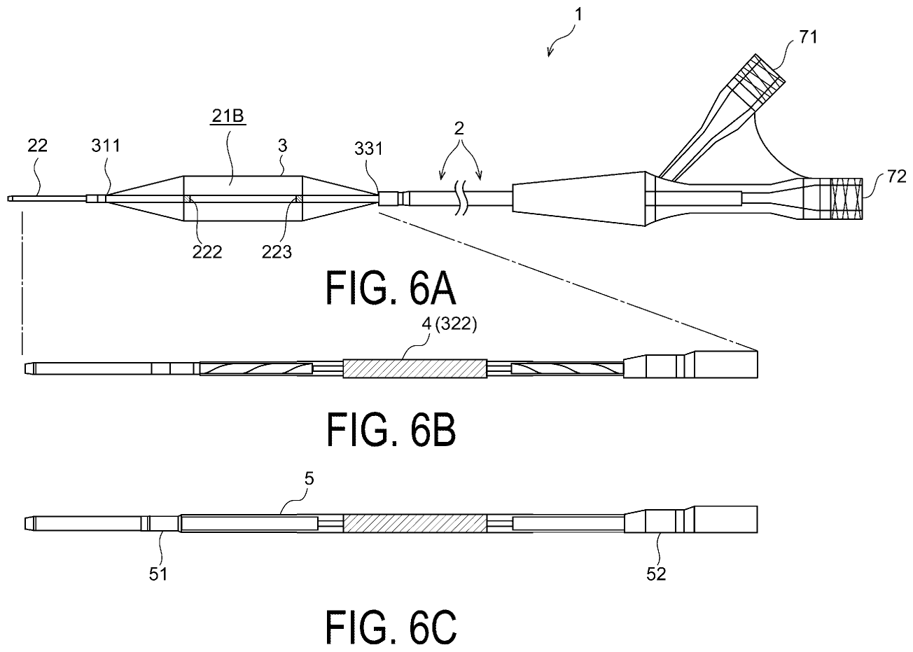

FIGS. 6A to 6C schematically illustrate a method of manufacturing the balloon catheter 1. In FIG. 6A, the balloon 3 is attached to the outer periphery on the distal end side of the shaft 2. The distal end portion 311 of the balloon 3 is bonded to the outer periphery of the guidewire tube 22 of the shaft 2, and is tightly closed to prevent the expansion fluid in the balloon 3 (internal space 21B) from leaking. The proximal end portion 331 of the balloon 3 is bonded to the outer periphery of the shaft 2 with the expansion fluid tube 21 and guidewire tube 22 as in FIG. 4A and is sealed such that the expansion fluid of the balloon 3 (internal space 21B) does not leak. While the guidewire tube 22 of the shaft 2 is formed through the entire length of the balloon 3, the expansion fluid tube 21 terminates within the internal space 21B of the balloon 3, and thus the expansion fluid can flow into and out from the internal space 21B of the balloon 3 through the opening end of the expansion fluid tube 21. The balloon 3 having the both end portions 311 and 331 bonded to the outer periphery of the shaft 2 as described above is wound around the outer periphery of the shaft 2 to be folded into a form of a plurality of (three for example) vanes.

In FIG. 6B, the elastic band 4 is attached to the center portion 322 of the folded balloon 3. For example, the distal end of the guidewire tube 22 is inserted in the elastic band 4, and the elastic band 4 slides to the center portion 322 of the balloon 3. In this process, the elastic band 4 does not need to be bonded to the outer periphery of the balloon 3 (center portion 322). A lubricant such as silicon oil or silicon powder may be added or applied between the outer periphery of the balloon 3 and the inner periphery of the elastic band 4 for the sake of better ease of assembly or the like. In FIG. 6C, the covering member 5 having an elongated tube shape is attached to cover the folded balloon 3 and the elastic band 4, and has the distal end portion and the proximal end portion fixed by the fixing tube 51 and the fixing tube 52. In this process, the covering member 5 does not need to be bonded to a portion of the balloon 3 other than the distal end portion 311 and the proximal end portion 331, and to the outer periphery of the elastic band 4. A lubricant such as silicon oil or silicon powder may be added or applied between the outer periphery of the elastic band 4 and the inner periphery of the covering member 5 for the sake of better ease of assembly or the like.

The elastic band 4 and the covering member 5 both having elasticity are provided as separate members in the embodiment as illustrated in FIG. 4B, but may be integrally formed by the same elastic material. With such an elastic member, the function of the elastic band 4 is achieved with the thickness at the position corresponding to the center portion 322 of the balloon 3 set to be larger than those in other portions.

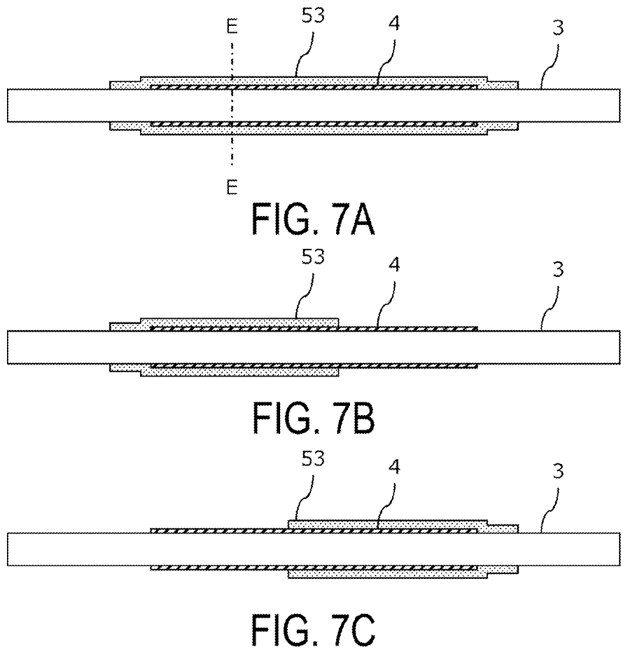

FIGS. 7A to 7C are cross-sectional views schematically illustrating a plurality of modifications of the covering member 5. These covering members 5 are a coating material 53 that covers at least part of the boundary between the elastic band 4 (securing member) and the outer periphery of the balloon 3. Any material can be used for the coating material 53. For example, the coating material 53 is formed by any elastic material such as polyurethane as in the embodiment described above. The coating material in a fluid or liquid form is applied in a desired portion of the outer periphery of the elastic band 4 and the balloon 3 and then is dried. As a result, a coating layer (coating material 53) serving as the covering member 5 illustrated is formed over both of the elastic band 4 and the balloon 3 in the axial direction. Alternatively, the balloon 3 to which the elastic band 4 is attached may be dipped (put in) into the coating material in a fluid or liquid form and then dried to form the coating layer (coating material 53) as the covering member 5.

The coating material 53 covers at least one of: at least part of the boundary between the balloon 3 and the distal end side of the elastic band 4 (left end side in FIG. 7); and at least part of the boundary between the balloon 3 and the proximal end side (right end side in FIG. 7) of the elastic band 4. Specifically, the coating material 53 may be formed to cover the boundary between the balloon 3 and the distal end side and the proximal end side of the elastic band 4 as illustrated in FIG. 7A, may be formed to cover only the boundary between the balloon 3 and the distal end side of the elastic band 4 as illustrated in FIG. 7B, or may be formed to cover only the boundary between the balloon 3 and the proximal end side of the elastic band 4 as illustrated in FIG. 7C. In any of the cases, the elastic band 4 and the balloon 3 are fixed via the coating material 53, and thus the elastic band 4 can be effectively prevented from being displaced from the predetermined position (center portion 322) of the balloon 3. To reliably provide this displacement prevention function, the coating material 53 preferably covers half the length of the elastic band 4 in the axial direction (left and right direction in FIG. 7) as illustrated in FIG. 7A to FIG. 7C.

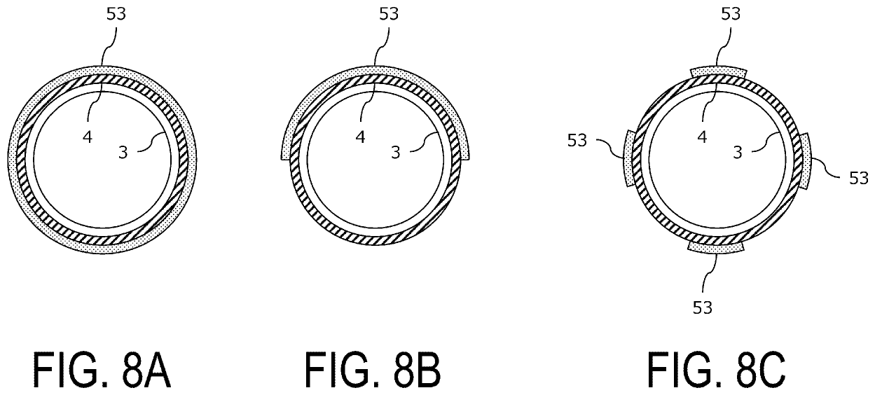

FIGS. 8A to 8C schematically illustrate a plurality of examples of a cross section taken along line E-E in FIG. 7. In the figure, only the balloon 3, the elastic band 4, and the coating material 53 (covering member 5) are illustrated, and other configurations inside the balloon 3 and the like are omitted. As illustrated in FIG. 8A, the coating material 53 may cover the entire outer periphery of the elastic band 4 and/or balloon 3. As illustrated in FIG. 8B, the coating material 53 may cover only part of the outer periphery of the elastic band 4 and/or balloon 3. In the example of FIG. 8B, the coating material 53 covers half (upper half) of the outer periphery of the elastic band 4 and/or the balloon 3, and the remaining half (lower half) is not provided with the coating material 53 and thus is exposed. Note that the length of the coating material 53, in the circumferential direction, covering the outer periphery of the elastic band 4 and/or the balloon 3 is not limited to the half the periphery as in FIG. 8B, and may be larger than half the periphery, or may be smaller than half the periphery.

As illustrated in FIG. 8B, with the coating material 53 not covering the entire outer periphery of the elastic band 4 and/or the balloon 3, there is an advantage that the expansion of the balloon 3 and the elastic band 4 is less likely to be inhibited by the coating material 53. The coating material 53 covering only part of the outer periphery of the balloon 3 and the elastic band 4 may be separated, ruptured, or break when the balloon 3 and the elastic band 4 expand from the inner side, but this facilitates (does not inhibit) the expansion of the balloon 3 and the elastic band 4. The coating material 53 intentionally losing its function when the balloon 3 and the elastic band 4 expand, is specialized for the function of preventing the displacement of the balloon 3 folded in the completely compressed state and the elastic band 4 before expansion. As illustrated in FIG. 8C, with a plurality of the coating materials 53 provided at a plurality of different positions in the circumferential direction of the elastic band 4 and/or the balloon 3 while being separated from each other, the coating material 53 can more easily lose its function when the balloon 3 and the elastic band 4 expand.

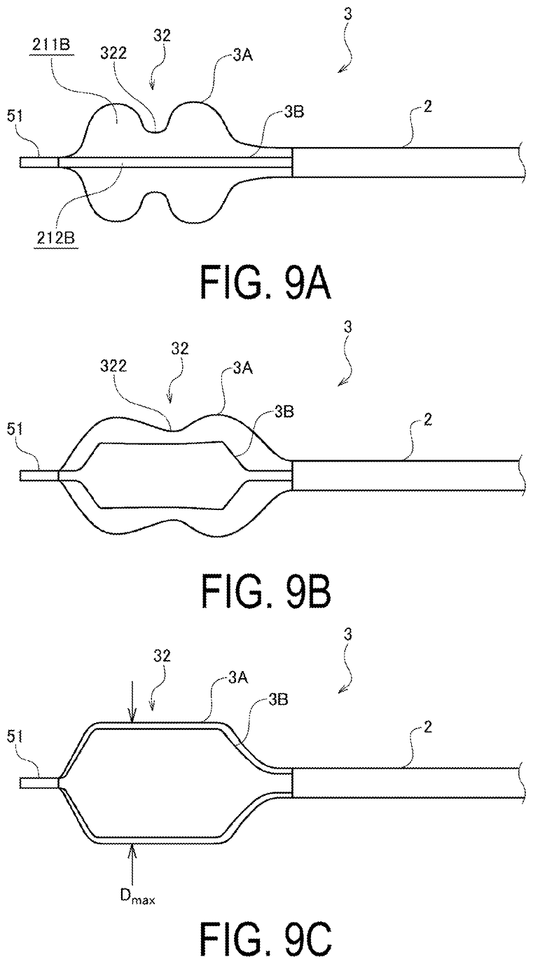

FIGS. 9A to 9C schematically illustrate a modification of the balloon 3. In this figure, only the shaft 2, the balloon 3, and the fixing tube 51 that is practically the distal end of the balloon 3 are illustrated, and other configurations inside and outside the balloon 3 are omitted. The balloon 3 according to the present modification is attached to the distal end side of the shaft 2 (left side in FIG. 9), and includes a first balloon 3A and a second balloon 3B that can be expanded by an expansion fluid supplied from the proximal end side (right side in FIG. 9) of the shaft 2. The first balloon 3A is provided closer to the outer side than the second balloon 3B in the expansion direction, and the second balloon 3B is provided closer to the inner side than the first balloon 3A in the expansion direction. Thus, the balloon 3 has a dual structure achieved with the first balloon 3A on the outer side and the second balloon 3B on the inner side.

As illustrated in FIG. 9A, the first balloon 3A on the outer side expands before the second balloon 3B on the inner side does. Specifically, the first balloon 3A is formed by a material softer than the second balloon 3B. Thus, even when the expansion fluid is supplied to the first balloon 3A and the second balloon 3B from the balloon expansion lumen 21A at substantially the same timing, the first balloon 3A expands before the second balloon 3B does. For example, the soft first balloon 3A on the outer side is formed by urethane or the like, and the hard second balloon 3B on the inner side is formed by nylon or the like. The first balloon 3A may have a smaller thickness than the second balloon 3B to be softer than the second balloon 3B. This expression “the first balloon 3A is softer than the second balloon 3B” means that the first balloon 3A is expanded by a larger amount than the second balloon 3B and/or that the expansion start timing of the first balloon 3A is earlier than that of the second balloon 3B, under a condition that the pressure of the expansion fluid supplied to the balloons is the same. The hardness of the first balloon 3A and the second balloon 3B may be irrelevant, and the first balloon 3A may expand before the second balloon 3B does, by first supplying the expansion fluid to the first balloon 3A from the first balloon expansion lumen, and then to the second balloon 3B from the second balloon expansion lumen.

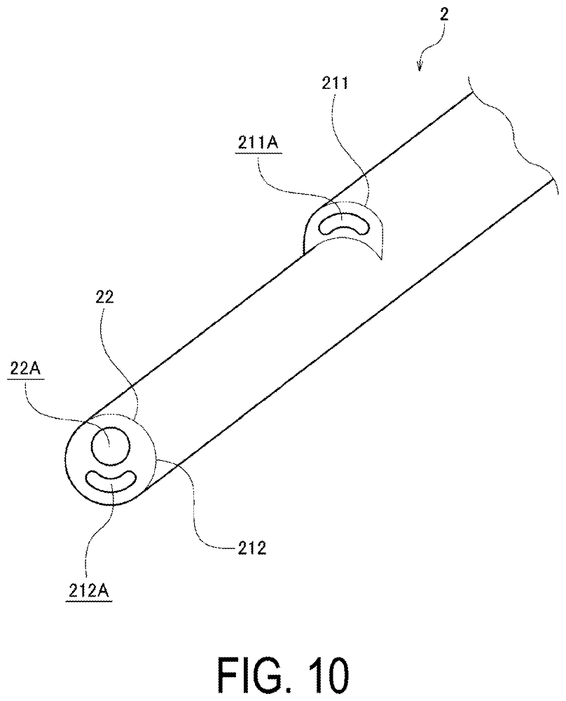

FIG. 10 is a perspective view schematically illustrating a modification of a shaft 2 with which expansion fluid can be supplied to the first balloon 3A and the second balloon 3B at the same timing or different timings. In this figure, configurations (including balloon 3) other than the shaft 2 are omitted. The shaft 2 includes the expansion fluid tube 21 and the guidewire tube 22 as described above with reference to FIGS. 4A to 4D and the like. Still, the shaft 2 is different from that illustrated in FIGS. 4A to 4D in that the expansion fluid tube 21 is divided into a first expansion fluid tube 211 and a second expansion fluid tube 212. The balloon expansion lumen 21A illustrated in FIGS. 4A to 4D and the like is divided into a first balloon expansion lumen 211A formed in the first expansion fluid tube 211 and a second balloon expansion lumen 212A formed in the second expansion fluid tube 212.

The distal end side opening end of the first expansion fluid tube 211 forming the first balloon expansion lumen 211A is inserted in an internal space 211B of the first balloon 3A (FIG. 9). The internal space 211B of the first balloon 3A is a space defined by an inner peripheral surface and the outer peripheral surface of the first balloon 3A on the outer side and the second balloon 3B on the inner side of the balloon 3 having the dual structure. The distal end side opening end of the second expansion fluid tube 212 forming the second balloon expansion lumen 212A is inserted in an internal space 212B of the second balloon 3B (FIG. 9). The internal space 212B of the second balloon 3B is a space defined by the inner peripheral surface of the second balloon 3B. Conversely, the opening end of the first expansion fluid tube 211 may be inserted in the internal space 212B of the second balloon 3B on the inner side, and the opening end of the second expansion fluid tube 212 may be inserted in the internal space 211B of the first balloon 3A on the outer side.

In the shaft 2 of the example illustrated in FIG. 10, the opening end of the first expansion fluid tube 211 and the opening end of the second expansion fluid tube 212 are at different positions in the axial direction. Specifically, the opening end of the first expansion fluid tube 211 is provided at the axial direction position of the balloon 3 on the proximal end side (the position of the proximal end side tapered portion 33 for example). The opening end of the second expansion fluid tube 212 is provided at the axial direction position of the balloon 3 on the distal end side (the position of the distal end side tapered portion 31 for example). The distal end of the guidewire tube 22 illustrated to be at the same position as the opening end of the second expansion fluid tube 212 in FIG. 10 is actually positioned at the distal end (opening end 221) of the balloon catheter 1 as described above with reference to FIG. 3 and the like. The opening end of the first expansion fluid tube 211 and the opening end of the second expansion fluid tube 212 may be at the same axial direction position.

As illustrated in FIG. 9A, the intermediate portion 32 of the first balloon 3A on the outer side that expands before the second balloon 3B on the inner side does may be expand into a constricted shape having a constricted portion in the center portion 322 instead of the straight tube shape as illustrated in FIG. 6A. For example, the intermediate portion 32 of the first balloon 3A is formed (manufactured) to have an original shape with constriction. Thus, the first balloon 3A naturally expands into the constricted shape when the expansion fluid is supplied from the first balloon expansion lumen 211A to the internal space 211B. When the first balloon 3A is expanded as illustrated in FIG. 9A in a state where the center portion 322 is in the vicinity of the papilla 91 (FIG. 1) that is the dilatation target, the distal end portion and the proximal end portion having a larger diameter than the constricted portion (center portion 322) supports the papilla 91 from both sides. Thus, the constricted portion (center portion 322) of the first balloon 3A can be reliably positioned with respect to the papilla 91 that is the dilatation target part.

The intermediate portion 32 of the first balloon 3A may expand into the straight tube shape as illustrated in FIG. 6A. As described above, the first balloon 3A is formed by a material softer than the second balloon 3B, and thus can deform and expand to conform with the shape of the dilatation target part such as the papilla 91, even when the first balloon 3A expands into the straight tube shape. As a result, the first balloon 3A naturally forms a constricted shape similar to that in FIG. 9A to support the papilla 91 or the like from both sides. Thus, the first balloon 3A expanding into the straight tube shape is also positioned with the constricted portion naturally formed at the position of the papilla 91 and the like.

In FIG. 9B illustrating a state after that in FIG. 9A, the first balloon 3A on the outer side continues to be expanded through the first balloon expansion lumen 211A, and the second balloon 3B on the inner side substantially starts to be expanded through the second balloon expansion lumen 212A. As described above, there is a difference in expansion start timing between the first balloon 3A and the second balloon 3B because the second balloon 3B is harder than the first balloon 3A and/or because the timing at which the expansion fluid starts to be supplied to the second balloon 3B is later than the timing at which the expansion fluid starts to be supplied to the first balloon 3A. The intermediate portion 32 of the second balloon 3B on the inner side expands into the straight tube shape as in FIG. 6A. The constricted portion formed in the intermediate portion 32 (center portion 322) of the first balloon 3A on the outer side shrinks due to the increased pressure of the expansion fluid continuously supplied to the first balloon 3A and the pressure in the expansion direction from the second balloon 3B expanding into the straight tube shape from the inner side of the first balloon 3A.

In FIG. 9C illustrating the state after that in FIG. 9B, the expansion of the second balloon 3B on the inner side through the second balloon expansion lumen 212A substantially ends. In the final expanded state of the second balloon 3B, the intermediate portion 32 thereof has expanded into the straight tube shape with the maximum expansion diameter Dmax as in FIG. 5C. The intermediate portion 32 of the soft first balloon 3A on the outer side has followed the hard second balloon 3B to deform into the straight tube shape having a diameter that is substantially the same as the maximum expansion diameter Dmax. Thus, the constricted portion formed in the intermediate portion 32 (center portion 322) of the first balloon 3A on the outer side substantially does not exist in the final expanded state of the second balloon 3B on the inner side. In this state, the dilatation target part such as the papilla 91 held by the constricted portion (center portion 322) of the soft first balloon 3A on the outer side is dilated (spreads out) effectively by the hard second balloon 3B on he inner side.

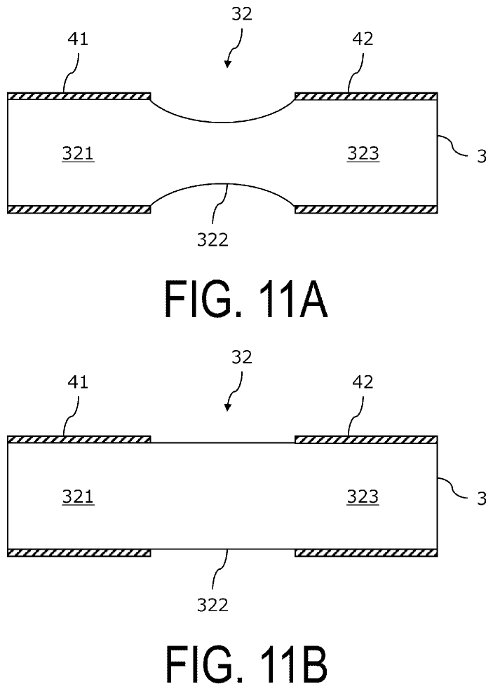

FIGS. 11A and 11B are cross-sectional views schematically illustrating a modification of the balloon catheter 1. In the figure, only the intermediate portion 32 of the balloon 3 and securing bands 41 and 42 described below are illustrated, and other configurations will be omitted. The balloon 3 according to the present modification includes a distal end side securing band 41 and a proximal end side securing band 42 serving as an end portion securing structure and an end portion restricting member, respectively, that limit the expansion in the distal end portion (including the distal end side straight tube portion 321) and the proximal end portion (including the proximal end side straight tube portion 323) after the expansion into a constricted shape with the constricted portion being in the intermediate portion 32 (the center portion 322 in particular) between the distal end portion and the proximal end portion.

As illustrated in FIG. 11A, the center portion 322 of the balloon 3 expands into the constricted shape with the constricted portion, instead of the straight tube shape as illustrated in FIG. 6A. Specifically, the center portion 322 of the balloon 3 is formed (manufactured) to have an original shape with constriction. Thus, the center portion 322 of the balloon 3 naturally expands into the constricted shape when the expansion fluid is supplied from the balloon expansion lumen 21A to the internal space 21B. When the balloon 3 is expanded as illustrated in FIG. 11A in a state where the center portion 322 is in the vicinity of the papilla 91 (FIG. 1) that is the dilatation target, the distal end portion (the distal end side straight tube portion 321 in particular) and the proximal end portion (the proximal end side straight tube portion 323 in particular) having a larger diameter than the constricted portion (center portion 322) supports the papilla 91 from both sides. Thus, the constricted portion (center portion 322) of the balloon 3 can be reliably positioned with respect to the papilla 91 that is the dilatation target part.

The distal end side securing band 41 wound around the outer periphery of the distal end side straight tube portion 321 closer to the distal end side (left side in FIG. 11) than the constricted center portion 322 limits the expansion of the balloon 3 in the distal end side straight tube portion 321. Similarly, the proximal end side securing band 42 wound around the outer periphery of the proximal end side straight tube portion 323 closer to the proximal end side (right side in FIG. 11) than the constricted center portion 322 limits the expansion of the balloon 3 in the proximal end side straight tube portion 323. Even when the pressure of the expansion fluid in the balloon 3 rises from that in FIG. 11A to that in FIG. 11B, in the distal end side straight tube portion 321, the distal end side securing band 41 limits the expansion and thus the diameter hardly increases from that in FIG. 11A. In the proximal end side straight tube portion 323, the proximal end side securing band 42 limits the expansion, and thus the diameter hardly increases from that in FIG. 11A.

On the other hand, the center portion 322 without the securing bands 41 and 42 expands from the state in FIG. 11A to the state in FIG. 11B, to have the same diameter as the distal end side straight tube portion 321 and the proximal end side straight tube portion 323. As a result, as illustrated in FIG. 11B, the intermediate portion 32 has a substantially straight tube shape entirely over the distal end side straight tube portion 321, the center portion 322, and the proximal end side straight tube portion 323. Thus, the constricted portion formed in the center portion 322 in FIG. 11A substantially does not exist in the final expanded state of the balloon 3 in FIG. 11B. In this state, the dilatation target part such as the papilla 91 held by the constricted portion in the center portion 322 in FIG. 11A is effectively dilated (spread out) by the center portion 322 in the substantially straight tube shape in FIG. 11B.

According to the present modification, the balloon 3 can be reliably positioned with respect to the dilatation target part, with the constricted portion formed in the intermediate portion 32 (center portion 322) at the time when the balloon 3 expands. When the constricted portion is further expanded by the expansion fluid to dilate the dilatation target part, the distal end side securing band 41 and the proximal end side securing band 42 serving as the end portion securing structure effectively prevent the distal end portion (distal end side straight tube portion 321) and the proximal end portion (proximal end side straight tube portion 323) on the distal and proximal end sides of the constricted portion from excessively expanding.

The end portion securing structure is not limited to the securing bands 41 and 42 wound around the outer periphery of the straight tube portions 321 and 323, and may be formed by a distal end portion (including the distal end side straight tube portion 321) and the proximal end portion (including the proximal end side straight tube portion 323) being harder than the intermediate portion 32 (the center portion 322 in particular). Similar to the meaning of the term “soft” described above with reference to FIG. 9, this term “hard” indicates that the amount of expansion of the distal end portion and the proximal end portion supplied with the expansion fluid at the same pressure is smaller than that of the intermediate portion 32 (the center portion 322 as the constricted portion). Thus, the “soft” constricted portion (center portion 322) can easily expand when the pressure of the expansion fluid rises, as in the transition from FIG. 11A to FIG. 11B. On the other hand, the “hard” distal end portion (distal end side straight tube portion 321) and proximal end portion (proximal end side straight tube portion 323) hardly expand even when the pressure of the expansion fluid rises, as in the transition from FIG. 11A to FIG. 11B. The distal end side straight tube portion 321 and the proximal end side straight tube portion 323 “harder” than the center portion 322 may be formed by a material (nylon for example) harder than the center portion 322 (urethane for example), may be obtained with a thickness larger than that of the center portion 322, or may be obtained with a multilayer balloon with a larger number of layers than the center portion 322.

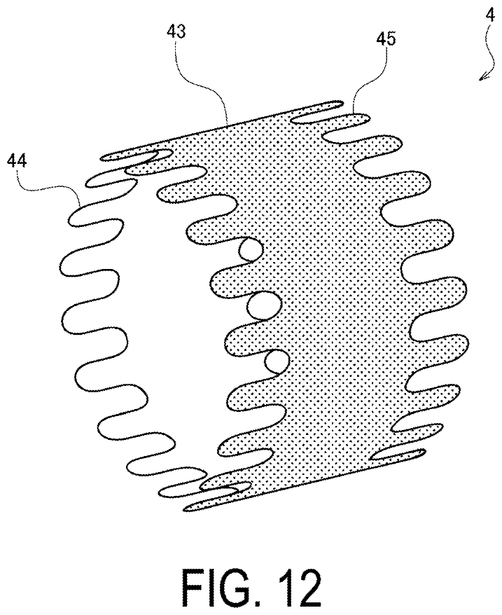

FIG. 12 schematically illustrates a modification of the elastic band 4. In the figure, only the elastic band 4 is illustrated, and other configurations are omitted. The elastic band 4 according to the present modification is a securing member that is wound around the outer periphery of the intermediate portion 32 (center portion 322 in particular) between the distal end portion and the proximal end portion of the balloon 3 to limit the expansion of the balloon 3 in the intermediate portion 32, and includes a securing relaxation structure that relaxes the restriction in at least one of the distal end portion and the proximal end portion. In the example illustrated in FIG. 12, a distal end side securing relaxation structure 44 and a proximal end side securing relaxation structure 45 having a recessed and protruding shape facing the axial direction are formed along the circumferential direction of an annular and band shaped band body 43 of the elastic band 4, on both the distal end side and the proximal end side of the band body 43. The recessed and protruding shape of the securing relaxation structures 44 and 45 is formed by a plurality notches in the axial direction (recesses of the recessed and protruding shape) formed along the circumferential direction in the distal end portion and the proximal end portion of the annular and band shaped elastic band 4 illustrated in FIG. 3 and the like. The notches in the axial direction forming the securing relaxation structures 44 and 45 may be slits or cuts in the axial direction.

With such securing relaxation structures 44 and 45, securing in the end portions of the elastic band 4 is relaxed compared with the securing in the center portion (band body 43). As a result, the end portions of the elastic band 4 where the securing relaxation structures 44 and 45 are formed expand beyond the band body 43 in the center portion. Thus, a minute constricted portion is formed in the elastic band 4 in addition to the constricted portion in the balloon 3, whereby the constricted elastic band 4 (and the balloon 3) can be reliably positioned with respect to the dilatation target part such as a constricted part. The securing relaxation structure in the end portions in the elastic band 4 may be any structure achieving gradation (reduction) of the securing force relative to that in the center portion. For example, the elastic band 4 is formed by an end portion band (first securing member) provided in the distal end portion and/or the proximal end portion, and a center portion band (second securing member) provided in the remaining portion that is the center portion. With the width in the axial direction and the thickness in the radial direction of the end portion band set to be smaller than that of the center portion band (corresponding to the band body 43), the securing force of the end portion band can be made smaller than that of the center portion band, whereby the securing relaxation structure (end portion band) is formed in the end portion of the elastic band 4.

The present disclosure has been described above based on the embodiments. It should be understood by those skilled in the art that the embodiments are examples, that various modifications are possible in the combination of components and processing operations, and that such modifications are also within the scope of the present disclosure.

The present disclosure may be expressed by the following items. Note that, regardless of the dependent relationship between the items described as example below, depending on any combination of elements of each the described embodiments and modifications, elements of the items can be freely combined without contradicting each other.
Item 1:

A catheter comprising:

a shaft to be inserted into a body;

a balloon that is attached to the shaft on a distal end side and is expandable by a fluid supplied from a side of a proximal end of the shaft;

a securing member that is wound around an outer periphery of an intermediate portion between a distal end portion and a proximal end portion of the balloon, and is configured to limit expansion of the balloon at the intermediate portion; and

a covering member that covers an outer periphery of the securing member.
Item 2:

The catheter according to item 1, wherein the securing member is elastically deformable in response to the expansion of the balloon.
Item 3:

The catheter according to item 1 or 2, wherein a secured diameter of the securing member is smaller than a maximum expansion diameter of the intermediate portion of the balloon.
Item 4:

The catheter according to any one of items 1 to 3, wherein, in a state where pressure of the fluid is lower than a predetermined value, an expansion diameter of the securing member is smaller than a maximum expansion diameter of the intermediate portion of the balloon by at least 20%.
Item 5:

The catheter according to item 4, wherein

the balloon is expandable to the maximum expansion diameter from a folded state, in accordance with pressure of the fluid,

the securing member is expandable beyond the secured diameter, by being elastically deformed by the balloon expanding, and

the covering member is expandable by being elastically deformed by the balloon or the securing member expanding.
Item 6:

The catheter according to any one of items 1 to 5, wherein the securing member has a higher elastic modulus than the covering member.
Item 7:

The catheter according to item 6, wherein the balloon has a higher elastic modulus than the securing member.
Item 8:

The catheter according to any one of items 1 to 7, wherein the securing member is an annular member wound around the outer periphery of the intermediate portion.
Item 9:

The catheter according to any one of items 1 to 8, wherein the securing member is a spiral member wound around the outer periphery of the intermediate portion.
Item 10:

The catheter according to any one of items 1 to 9, wherein the covering member covers part of outer periphery of the balloon around which the securing member is not wound, as well as the outer periphery of the securing member.
Item 11:

The catheter according to item 10, wherein the covering member covers part of outer periphery of the shaft where the balloon is not attached, as well as the outer periphery of the securing member and the part of the outer periphery of the balloon.
Item 12:

The catheter according to any one of items 1 to 11, wherein the covering member is longer than the securing member in a direction connecting the distal end and the proximal end of the shaft.
Item 13:

The catheter according to item 12, wherein the covering member is longer than the balloon in the direction connecting the distal end and the proximal end of the shaft.
Item 14:

The catheter according to any one of items 1 to 13, wherein a length of the securing member is from 1 mm to 10 mm in a direction connecting the distal end and the proximal end of the shaft.
Item 15:

The catheter according to item 14, wherein

the intermediate portion of the balloon is a straight tube portion having a maximum expansion diameter that is substantially uniform,

a length of the straight tube portion in the direction connecting the distal end and the proximal end of the shaft is from 10 mm to 40 mm.
Item 16:

The catheter according to any one of items 1 to 15, wherein a length of the intermediate portion of the balloon is two to eight times longer than a length of the securing member in the direction connecting the distal end and the proximal end of the shaft.
Item 17:

The catheter according to any one of items 1 to 16, wherein the covering member is fixed to parts of outer periphery of the catheter that are closer to a distal end side and a proximal end side than the securing member.
Item 18:

The catheter according to any one of items 1 to 17, wherein the securing member is bonded to none of the balloon and the covering member.
Item 19:

The catheter according to any one of items 1 to 18, wherein the covering member is a coating material that covers at least part of a boundary between the securing member and the outer periphery of the balloon.
Item 20:

The catheter according to item 19, wherein the coating material covers at least part the boundary between the balloon and the distal end side of the securing member and at least part of the boundary between the balloon and the proximal end side of the securing member.
Item 21:

The catheter according to item 19 or 20, wherein the coating material covers half the length or more of the securing member, in the direction connecting the distal end and the proximal end of the shaft.
Item 22:

The catheter according to any one of items 19 to 21, wherein a plurality of the coating materials are provided at a plurality of different positions in a circumferential direction of the securing member and the balloon while being separated from each other.
Item 23:

A method of expanding a balloon in a catheter including a shaft to be inserted into a body, the balloon that is attached to the shaft on a distal end side and is expandable by a fluid supplied from a side of a proximal end of the shaft, a securing member that is wound around an outer periphery of an intermediate portion between a distal end portion and a proximal end portion of the balloon, and is configured to limit expansion of the balloon in an intermediate portion, and a covering member that covers an outer periphery of the securing member, the method comprising reducing, when the balloon is expanded together with the securing member and the covering member by supplying a fluid into the balloon, a rate of expansion of the intermediate portion around which the securing member is wound.
Item 24:

A catheter comprising:

a shaft to be inserted into a body;

a balloon that is attached to the shaft on a distal end side and is expandable by a fluid supplied from a side of a proximal end of the shaft; and

a covering member that covers an outer periphery of the balloon, and has a large thickness at an intermediate portion between a distal end portion and a proximal end portion of the balloon compared with other portions.
Item 25:

A catheter comprising:

a shaft to be inserted into a body;

a balloon that is attached to the shaft on a distal end side and is expandable by a fluid supplied from a side of a proximal end of the shaft; and

a visually recognized portion that is provided in an intermediate portion between a distal end portion and a proximal end portion of the balloon, and is visually distinguishable from other portions of the balloon.
Item 26:

The catheter according to item 25 further comprising a securing member that is wound around an outer periphery of the intermediate portion and is configured to limit expansion of the balloon at the intermediate portion, wherein

the visually recognized portion is formed by the securing member that is visually distinguishable from the balloon.
Item 27:

The catheter according to item 25 or 26, wherein the visually recognized portion is different from the other portions of the balloon in color and/or pattern.
Item 28:

A catheter comprising:

a shaft to be inserted into a body; and

a balloon that is attached to the shaft on a distal end side and includes a first balloon and a second balloon expandable by a fluid supplied from a side of a proximal end of the shaft, the first balloon being provided closer to an outer side in a direction of the expansion than the second balloon, and expanding before the second balloon does.
Item 29:

The catheter according to 28, wherein the first balloon is softer than the second balloon. Item 30:

The catheter according to item 28 or 29, wherein

the first balloon is expanded by the fluid into a constricted shape having a constricted portion in an intermediate portion between a distal end portion and a proximal end portion of the balloon, and

the second balloon is expanded by the fluid into a straight tube shape in the intermediate portion.
Item 31:

The catheter according to item 30, wherein the constricted portion of the first balloon that has been expanded into the constricted shape by the fluid is reduced by the second balloon expanded by the fluid into the straight tube shape on inner side in the direction of the expansion.
Item 32:

A catheter comprising:

a shaft to be inserted into a body; and

a balloon that is attached to the shaft on a distal end side, is expandable by a fluid supplied from a side of a proximal end of the shaft, and includes an end portion securing structure that limits, after expansion into a constricted shape having a constricted portion in an intermediate portion between a distal end portion and a proximal end portion, the expansion in the distal end portion and the proximal end portion.
Item 33:

The catheter according to item 32, wherein the end portion securing structure is formed by an end portion restricting member that is wound around an outer periphery of the distal end portion and the proximal end portion, and limits the expansion of the balloon at the distal end portion and the proximal end portion.
Item 34:

The catheter according to item 32 or 33, wherein the end portion securing structure is formed by the distal end portion and the proximal end portion being harder than the intermediate portion.
Item 35:

A catheter comprising:

a shaft to be inserted into a body; and

a balloon that is attached to the shaft on a distal end side, is expandable by a fluid supplied from a side of a proximal end of the shaft, and includes a compression structure configured to cause an intermediate portion between a distal end portion and a proximal end portion to compress before the distal end portion and the proximal end portion compress, at a time of compression in which the fluid is discharged.
Item 36:

The catheter according to item 35, wherein the compression structure is formed by a securing member that is wound around an outer periphery of the intermediate portion and has elasticity to limit expansion of the balloon at the intermediate portion.
Item 37:

A method of compressing a balloon of a catheter including a shaft to be inserted into a body, and the balloon that is attached to the shaft on a distal end side, is expandable by a fluid supplied from a side of a proximal end of the shaft, and includes a compression structure configured to cause an intermediate portion between a distal end portion and a proximal end portion to compress before the distal end portion and the proximal end portion compress, at a time of compression in which the fluid is discharged, the method comprising increasing a rate of compression of the intermediate portion provided with the compression structure, when compressing the balloon by discharging the fluid in the balloon.
Item 38:

A catheter comprising:

a shaft to be inserted into a body;

a balloon that is attached to the shaft on a distal end side and is expandable by a fluid supplied from a side of a proximal end of the shaft; and

a securing member that is wound around an outer periphery of an intermediate portion between a distal end portion and a proximal end portion of the balloon, is configured to limit expansion of the balloon at the intermediate portion, and includes a securing relaxation structure configured to relax securing in at least one of a distal end portion and a proximal end portion of the securing member.
Item 39:

The catheter according to item 38, wherein the securing relaxation structure is formed by a notch formed in at least one of the distal end portion and the proximal end portion of the securing member.
Item 40:

The catheter according to item 38 or 39, wherein

the securing member includes a first securing member that is provided in at least one of a distal end portion and a proximal end portion of the securing member, and a second securing member that is provided in another portion, and

the securing relaxation structure is formed by the first securing member having lower securing force than the second securing member.

### REFERENCE SIGNS LIST

1 Balloon catheter
2 Shaft
3 Balloon
3A First balloon
3B Second balloon
4 Elastic band
5 Covering member
6 Guidewire
7 Manifold
10 Endoscope
11 Forceps channel
12 Camera
21 Expansion fluid tube
21A Balloon expansion lumen
21B Internal space
22 Guidewire tube
22A Wire lumen
31 Distal end side tapered portion
32 Intermediate portion
33 Proximal end side tapered portion
41 Distal end side securing band
42 Proximal end side securing band
43 Band body
44 Distal end side securing relaxation structure
45 Proximal end side securing relaxation structure
51 Fixing tube
52 Fixing tube
53 Coating material
71 Balloon expansion port
72 Guidewire port
90 Duodenum
91 Papilla
92 Common bile duct
93 Pancreatic duct
211 First expansion fluid tube
211A First balloon expansion lumen
211B Internal space
212 Second expansion fluid tube
212A Second balloon expansion lumen
212B Internal space
221 Opening end
222 Contrast marker
223 Contrast marker
311 Distal end portion
321 Distal end side straight tube portion
322 Center portion
323 Proximal end side straight tube portion
331 Proximal end portion

1. A catheter comprising:
a shaft to be inserted into a body;
a balloon that is attached to the shaft on a distal end side and is expandable by a fluid supplied from a side of a proximal end of the shaft;
a securing member that is wound around an outer periphery of an intermediate portion between a distal end portion and a proximal end portion of the balloon, and is configured to limit expansion of the balloon at the intermediate portion; and
a covering member that covers an outer periphery of the securing member.

2. The catheter according to claim 1, wherein the securing member is elastically deformable in response to the expansion of the balloon.

3. The catheter according to claim 1, wherein a secured diameter of the securing member is smaller than a maximum expansion diameter of the intermediate portion of the balloon.

4. The catheter according to claim 3, wherein
the balloon is expandable to the maximum expansion diameter from a folded state, in accordance with pressure of the fluid,
the securing member is expandable beyond the secured diameter, by being elastically deformed by the balloon expanding, and
the covering member is expandable by being elastically deformed by the balloon or the securing member expanding.

5. The catheter according to claim 1, wherein, in a state where pressure of the fluid is lower than a predetermined value, an expansion diameter of the securing member is smaller than a maximum expansion diameter of the intermediate portion of the balloon by at least 20%.

6. The catheter according to claim 1, wherein the securing member has a higher elastic modulus than the covering member.

7. The catheter according to claim 6, wherein the balloon has a higher elastic modulus than the securing member.

8. The catheter according to claim 1, wherein the securing member is an annular member wound around the outer periphery of the intermediate portion.

9. The catheter according to claim 1, wherein the covering member covers part of an outer periphery of the balloon around which the securing member is not wound, as well as the outer periphery of the securing member.

10. The catheter according to claim 9, wherein the covering member covers part of an outer periphery of the shaft where the balloon is not attached, as well as the outer periphery of the securing member and the part of the outer periphery of the balloon.

11. The catheter according to claim 1, wherein the covering member is longer than the securing member in a direction connecting the distal end and the proximal end of the shaft.

12. The catheter according to claim 11, wherein the covering member is longer than the balloon in the direction connecting the distal end and the proximal end of the shaft.

13. The catheter according to claim 1, wherein a length of the securing member is from 1 mm to 10 mm in a direction connecting the distal end and the proximal end of the shaft.

14. The catheter according to claim 13, wherein
the intermediate portion of the balloon is a straight tube portion having a maximum expansion diameter that is substantially uniform, and
a length of the straight tube portion in the direction connecting the distal end and the proximal end of the shaft is from 10 mm to 40 mm.

15. The catheter according to claim 1, wherein a length of the intermediate portion of the balloon is two to eight times longer than a length of the securing member in a direction connecting the distal end and the proximal end of the shaft.

16. The catheter according to claim 1, wherein the covering member is fixed to parts of an outer periphery of the catheter that are closer to a distal end side and a proximal end side than the securing member.

17. The catheter according to claim 1, wherein the securing member is bonded to none of the balloon and the covering member.

18. A catheter comprising:
a shaft to be inserted into a body; and
a balloon that is attached to the shaft at a distal end side, is expandable by a fluid supplied from a side of a proximal end of the shaft, and includes a compression structure configured to cause an intermediate portion between a distal end portion and a proximal end portion to compress before the distal end portion and the proximal end portion compress, at a time of compression in which the fluid is discharged.

19. The catheter according to claim 18, wherein the compression structure is formed by a securing member that is wound around an outer periphery of the intermediate portion and has elasticity to limit expansion of the balloon at the intermediate portion.

20. A catheter comprising:
a shaft to be inserted into a body;
a balloon that is attached to the shaft on a distal end side and is expandable by a fluid supplied from a side of a proximal end of the shaft; and
a covering member that covers an outer periphery of the balloon, and has a large thickness at an intermediate portion between a distal end portion and a proximal end portion of the balloon compared with other portions.
